(19) United States
(12) Patent Application Publication
(10) Pub. No.: US 2024/0318147 A1
(43) Pub. Date: Sep. 26, 2024

(54) RECOMBINANT ONCOLYTIC VIRUS, AND CONSTRUCTION METHOD THEREFOR AND USE THEREOF

(71) Applicant: REVOIMMUNE THERAPEUTICS INC., Shanghai (CN)

(72) Inventor: Kexing WU, Shanghai (CN)

(73) Assignee: REVOIMMUNE THERAPEUTICS INC., Shanghai (CN)

(21) Appl. No.: 18/266,457

(22) PCT Filed: Aug. 10, 2021
(86) PCT No.: PCT/CN2021/111761

§ 371 Date: Dec. 19, 2023

(30) Foreign Application Priority Data
Aug. 14, 2020 (CN) 202010816787.4

(51) Int. Cl. C12N 7/00; A61K 35/766; A61P 35/00

(52) U.S. Cl. CPC C12N 7/00; A61K 35/766; A61P 35/00; C12N 2760/20232

(57) ABSTRACT

Provided is a recombinant vesicular stomatitis oncolytic virus. The recombinant vesicular stomatitis oncolytic virus expresses a G protein, and the G protein has the following amino acid sequence: (a) SEQ ID NO: 1; (b) SEQ ID NO: 2; or (c) an amino acid sequence having at least 80% homology to (a) or (b).

### PRIORITY INFORMATION

The present application claims priority and benefit to a patent application having patent application Ser. No. 202010816787.4, filed on Aug. 14, 2020 to CNIPA, which is incorporated herein by reference in its entirety.

### INCORPORATION BY REFERENCE OF SEQUENCE LISTING

The sequence listing in the ASCII text file, named as 42192_SubstituteSequenceListing.txt of 9 KB, created on Dec. 19, 2023, and submitted to the United States Patent and Trademark Office via Patent Center, is incorporated herein by reference.

### FIELD

The present disclosure relates to the technical field of biomedicine, and more particularly, to a recombinant oncolytic virus, a method for constructing a recombinant virus, a method for improving a binding force of an oncolytic virus to a target cell, a pharmaceutical composition, and uses thereof.

### BACKGROUND

In the past decade, the mechanism by which oncolytic viruses can kill tumors by inducing the body's anti-tumor immune response has gradually become clear. Since German scientist, Jean Rommelaere, first called an oncolytic virus therapy as a tumor immunotherapy in 2011, oncolytic virus has now been accepted by the public as an important branch of tumor immunotherapy. Compared with other tumor immunotherapies, the oncolytic viruses have the advantages such as high killing efficiency, good targeting ability, small side effects, multiple tumor killing pathway to avoid the drug resistance, and low cost.

Due to the small genome of the virus, it is relatively easy to carry out various transformations through genetic engineering, and the transformation and packaging of the virus can be carried out by conventional means. These technologies are relatively mature and require relatively low cost. Therefore, it is easy to modify the oncolytic virus to specifically target cancer cells by utilizing the characteristics of the oncolytic virus itself as well as the differences between cancer cells and normal cells.

Because most cancer cells' own mechanisms for virus clearance are impaired (e.g., a protein kinase R (PKR), a key factor for virus clearance in normal cells, is missing in cancer cells), the viruses are more likely to replicate and spread in the cancer cells. In addition, in recent decades, with the continuous deepening of researches, scientists have used the differences in many signaling pathways and metabolisms in cancer cells and normal cells to continuously improve the targeting properties of the oncolytic viruses to tumors by screening specific virus species and modifying virus genomes, in order to reduce the harm to the normal cells and improve the safety. For example, the approved T-vec knocks out γ34.5 gene of HSV-1 (herpes simplex virus type 1), and the expression product of the γ34.5 gene can inhibit the virus clearance mechanism in normal cells. Thus, the virus in unable to replicate in the normal cells after the γ34.5 gene knockout. However, cancer cells lack the virus clearance mechanism, and thus the γ34.5 gene knockout does not affect the replication of virus in cancer cells. JX594 (Pexa-Vec), which is currently in stage III clinical trial, knocks out thymidine kinase (TK) gene of vaccinia viruses. Since the replication of the virus is related to the level of TK in cells, JX594 from which TK has been knocked out can only replicate in cancer cells with high TK activity, but cannot replicate in normal cells (the TK activity of normal cells is lower than that of cancer cells). In CG0070, an E2F-1 promoter is added in front of a gene E1A, which is replicated by an adenovirus director, and the E2F-1 promoter is regulated by a retinoblastoma inhibitor protein (Rb), which is missing in bladder cancer. Therefore, the deletion of Rb can activate the transcriptional activity of E2F-1, thereby expressing the E1A gene in bladder cancer cells, and virus can also be specifically replicated in the bladder cancer cells. Reolysin is an unmodified wild-type reovirus whose proliferation relies on the activation of a Ras signaling pathway, and thus it can only proliferate specifically in Ras-activated cancer cells.

However, the tumor therapy is in the current status that there is still a lack of a therapeutic method capable of improving the specificity of tumor killing (i.e., specific killing of tumor cells relative to normal non-tumor cells) as well as enhancing the broad spectrum of tumor therapy (i.e., suitable for multiple tumor therapies at the same time).

Therefore, it is urgent to develop a recombinant oncolytic virus with high tumor cell specificity and broad spectrum of tumor therapy.

### SUMMARY

The present disclosure aims to solve one of the technical problems in the related art at least to a certain extent. An object of the present disclosure is to provide a recombinant oncolytic virus simultaneously having high tumor cell specificity and/or broad spectrum of tumor therapy.

The present disclosure is made based on the Applicant's following findings: G protein is the coat protein of VSV virus. The Applicant has found that the G protein of some vesicular stomatitis viruses can achieve a high binding force of the vesicular stomatitis viruses to some tumor cells through specific receptors on the surfaces of the tumor cells. Since the expression of the specific receptors on the surfaces of these tumor cells are very low in normal cells, the vesicular stomatitis viruses carrying such G proteins have high specificity to tumor cells. In addition, due to high expression of the specific receptors on the surfaces of these tumor cells in a variety of tumor cells, the vesicular stomatitis viruses carrying such G proteins have an improved broad spectrum of tumor therapy, that is, they can function in a variety of tumor cells.

Therefore, in a first aspect, the present disclosure provides use of a virus protein in the construction of a recombinant oncolytic virus. According to an embodiment of the present disclosure, the protein is selected from: (a) SEQ ID NO: 1; (b) SEQ ID NO: 2; or (c) an amino acid sequence having at least 80% homology to (a) or (b). The recombinant oncolytic virus constructed using the protein has strong binding force and killing force to tumor cells.

According to an embodiment of the present disclosure, the above-mentioned use may further include at least one of the following additional technical features.
according to an embodiment of the present disclosure, the virus protein is derived from wild-type vesicular stomatitis viruses.

According to an embodiment of the present disclosure, the virus protein includes an amino acid sequence having at least 90%, at least 95%, at least 98%, or at least 99% homology to (a) or (b).

According to an embodiment of the present disclosure, the recombinant oncolytic virus carries no heterologous gene. According to the embodiment of the present disclosure, the term “heterologous gene” described herein refers to a gene that has not been reported in wild-type vesicular stomatitis viruses, unless otherwise specified. In other words, all the encoded proteins in the recombinant vesicular stomatitis viruses are expressed in the wild-type vesicular stomatitis viruses.

According to an embodiment of the present disclosure, each gene coding sequence of a genome of the recombinant oncolytic virus is selected from the wild-type vesicular stomatitis viruses.

According to an embodiment of the present disclosure, the virus protein has a ZDOCK score of a binding force to a cell receptor not lower than 1,800, for example, not lower than 1,900, not lower than 2,000, or not lower than 2,100.

According to an embodiment of the present disclosure, the cell receptor includes at least one selected from CHRNA5, SSTR5, KISS1R, HTR1D, or CCR8.

According to an embodiment of the present disclosure, the recombinant oncolytic virus further expresses at least one of nuclear protein, phosphate protein, matrix protein, and

RNA-dependent RNA polymerase.

According to an embodiment of the present disclosure, the genome of the recombinant oncolytic virus carries: a nucleic acid molecule encoding the nuclear protein, a nucleic acid molecule encoding the phosphate protein, a nucleic acid molecule encoding the matrix protein, and a nucleic acid molecule encoding the RNA-dependent RNA polymerase.

According to an embodiment of the present disclosure, at least one of the nucleic acid molecule encoding the nuclear protein, the nucleic acid molecule encoding the phosphate protein, the nucleic acid molecule encoding the matrix protein, and the nucleic acid molecule encoding the RNA-dependent RNA polymerase that are carried by the genome of the recombinant oncolytic virus is derived from a Mudd summer subtype virus strain of the vesicular stomatitis virus.

In a second aspect, the present disclosure provides a method for constructing a recombinant vesicular stomatitis virus. According to an embodiment of the present disclosure, the method includes: allowing an initial vesicular stomatitis virus to express a G protein, the G protein including the following amino acid sequence: (a) SEQ ID NO: 1; (b) SEQ ID NO: 2; or (c) an amino acid sequence having at least 80% homology to (a) or (b); optionally, the G protein is derived from wild-type vesicular stomatitis viruses; optionally, the G protein includes at least one of a G protein with GenBank index number of X03633.1 and a G protein with GenBank index number of DQ408670.1; and optionally, the G protein comprises an amino acid sequence having at least 90%, at least 95%, at least 98%, at least 99%, or at least 99% homology to (a) and (b). As described above, according to an embodiment of the present disclosure, the recombinant vesicular stomatitis virus expressing the above-mentioned G protein has stronger specific targeting ability to tumor cells, a broader spectrum of killing tumors, and a more significant killing effect.

According to an embodiment of the present disclosure, the above-mentioned method may further include at least one of the following additional technical features.

According to an embodiment of the present disclosure, the initial oncolytic stomatitis virus further expresses at least one of selected from a nuclear protein, a phosphate protein, a matrix protein, and an RNA-dependent RNA polymerase. The inventors have unexpectedly found that the recombinant virus, which is constructed by combining these proteins with at least one of the G protein with GenBank index number of X03633.1 and the G protein with GenBank index number of DQ408670.1, has stronger tumor killing activity. In the inventors' opinion, the mechanism thereof may be in that for tumor cells, a variety of protein combinations from different sources may trigger immune responses other than those of protein combinations from the same source, thereby further improving the killing effect on the tumor cells.

According to an embodiment of the present disclosure, the initial oncolytic stomatitis virus is allowed to carry a nucleic acid molecule encoding the nuclear protein, a nucleic acid molecule encoding the phosphate protein, a nucleic acid molecule encoding the matrix protein, or a nucleic acid molecule encoding the RNA-dependent RNA polymerase; and optionally, at least one of the nucleic acid molecule encoding the nuclear protein, the nucleic acid molecule encoding the phosphate protein, the nucleic acid molecule encoding the matrix protein, and the nucleic acid molecule encoding the RNA-dependent RNA polymerase is derived from a Mudd summer subtype virus strain of the vesicular stomatitis virus. Therefore, the expression efficiency of these proteins and the killing effect of the recombinant oncolytic virus on tumors may be further improved.

In a third aspect, the present disclosure provides a recombinant oncolytic virus which is constructed according to the foregoing method for constructing a recombinant vesicular stomatitis virus. The oncolytic virus according to the embodiments of the present disclosure has a more significant killing effect on tumors.

In a fourth aspect, the present disclosure provides a method for improving a binding force of a vesicular stomatitis virus to a target cell. According to an embodiment of the present disclosure, the method includes: allowing the vesicular stomatitis virus to express a G protein, the G protein including the following amino acid sequence: (a) SEQ ID NO: 1; (b) SEQ ID NO: 2; or (c) an amino acid sequence having at least 80% homology to (a) or (b). As described above, according to an embodiment of the present disclosure, by expressing the G protein described above, the binding force of the virus binding to the target cell through a cell surface receptor can be effectively enhanced.

According to an embodiment of the present disclosure, the above-mentioned method may further include at least one of the following additional technical features.

According to an embodiment of the present disclosure, the G protein is derived from wild-type vesicular stomatitis viruses.

According to an embodiment of the present disclosure, the G protein includes at least one of a G protein with GenBank index number of X03633.1 and a G protein with GenBank index number of DQ408670.1.

According to an embodiment of the present disclosure, the G protein includes an amino acid sequence having at least 90%, at least 95%, at least 98%, or at least 99% homology to (a) or (b).

According to an embodiment of the present disclosure, the initial vesicular stomatitis virus is allowed to carry a nucleic acid molecule encoding the G protein.

In a fifth aspect, the present disclosure provides a recombinant oncolytic virus, which is constructed by the above-mentioned method for improving the binding force of the oncolytic virus to a target cell. The oncolytic virus according to the embodiments of the present disclosure has stronger binding and targeting properties to tumors, and has more significant killing effect.

In a sixth aspect, the present disclosure provides a recombinant oncolytic virus having high affinity for tumor cells. According to an embodiment of the present disclosure, the recombinant oncolytic virus expresses a virus protein having high affinity for a cell receptor, the virus protein being selected from: (a) SEQ ID NO: 1; (b) SEQ ID NO: 2; or (c) an amino acid sequence having at least 80% homology to (a) or (b). According to an embodiment of the present disclosure, the recombinant vesicular stomatitis virus expressing the above virus protein has stronger specific targeting ability to tumor cells, and a more significant killing effect.

In addition, according to an embodiment of the present disclosure, the above-mentioned recombinant vesicular stomatitis virus may further include at least one of the following additional technical features.

According to an embodiment of the present disclosure, the virus protein includes an amino acid sequence having at least 90%, at least 95%, at least 98%, or at least 99% homology to (a) or (b).

According to an embodiment of the present disclosure, the recombinant vesicular stomatitis virus carries no heterologous gene. According to an embodiment of the present disclosure, the term “heterologous gene” described herein refers to a gene that has not been reported in wild-type vesicular stomatitis viruses, unless otherwise specified. Alternatively, in other words, all proteins encoded in the recombinant vesicular stomatitis viruses are expressed in the wild-type vesicular stomatitis viruses.

According to an embodiment of the present disclosure, a gene coding sequence of a genome of the recombinant vesicular stomatitis virus is selected from wild-type vesicular stomatitis viruses.

According to an embodiment of the present disclosure, the virus protein has a ZDOCK score of a binding force to a cell receptor not lower than 1,800, e.g., not lower than 1,900, not lower than 2,000, or not lower than 2,100. Those skilled in the art can understand that, by entering sequences of the G protein and the cell receptor, a binding force characterization parameter “ZDOCK score” between the G protein and the cell receptor can be easily obtained. The inventors have found that when the ZDOCK score is not lower than 1,800, a binding force of a virus carrying the G protein to a tumor cell carrying the corresponding receptor can be significantly improved. According to an embodiment of the present disclosure, the ZDOCK score may be determined by conventional software (for example, see Pierce B G, Hourai Y, Weng Z. (2011) Accelerating Protein Docking in ZDOCK Using an Advanced 3D Convolution Library. PLOS One 6(9): e24657).

According to an embodiment of the present disclosure, the virus protein includes at least one of a G protein with GenBank index number of X03633.1 and a G protein with GenBank index number of DQ408670.1. An amino acid sequence corresponding to the GenBank index number of DQ408670.1 is set forth in SEQ ID NO: 1, and an amino acid sequence corresponding to the GenBank index number of X03633.1 is set forth in SEQ ID NO: 2. The inventors of the present disclosure have unexpectedly found that the G protein with GenBank index number of X03633.1 and the G protein with GenBank index number of DQ408670.1 have a significantly stronger binding force to the receptors of tumor cells than other G proteins.

According to an embodiment of the present disclosure, the recombinant vesicular stomatitis virus further expresses at least one selected from a nuclear protein, a phosphate protein, a matrix protein, and an RNA-dependent RNA polymerase. The inventors have unexpectedly found that the recombinant virus, which is constructed by combining these proteins with at least one of the G protein with GenBank index number of X03633.1 and the G protein with GenBank index number of DQ408670.1, has stronger tumor killing activity. In the inventors' opinion, the mechanism thereof may be in that for tumor cells, a variety of protein combinations from different sources may trigger immune responses other than those of protein combinations from the same source, thereby further improving the killing effect on the tumor cells.

According to an embodiment of the present disclosure, the recombinant vesicular stomatitis virus carries a nucleic acid molecule encoding the nuclear protein, a nucleic acid molecule encoding the phosphate protein, a nucleic acid molecule encoding the matrix protein, or a nucleic acid molecule encoding the RNA-dependent RNA polymerase. Therefore, the expression efficiency of these proteins may be further improved.

According to an embodiment of the present disclosure, at least one of the nucleic acid molecule encoding the nuclear protein, the phosphate protein, the nucleic acid molecule encoding the matrix protein, and the nucleic acid molecule encoding the RNA-dependent RNA polymerase is derived from a Mudd summer subtype virus strain of the vesicular stomatitis virus. The inventors have unexpectedly found that the recombinant virus, which is constructed by combining these proteins with at least one of the G protein with GenBank index number of X03633.1 and the G protein with GenBank index number of DQ408670.1, has stronger tumor killing activity.

In a seventh aspect, the present disclosure provides use of the above-mentioned oncolytic virus in the prevention or treatment of a disease.

According to an embodiment of the present disclosure, the disease is selected from a cancer or a tumor.

According to an embodiment of the present disclosure, the disease is selected from at least one of lung cancer, gastric cancer, liver cancer, intestinal cancer, esophageal cancer, breast cancer, cervical cancer, malignant lymphoma, nasopharyngeal cancer, or leukemia.

In an eighth aspect, the present disclosure provides use of the pharmaceutical composition or the recombinant vesicular stomatitis virus described above in the preparation of a medicament, and the medicament is for use in the treatment or prevention of a cancer or a tumor.

According to an embodiment of the present disclosure, the cancer is selected from at least one of lung cancer, gastric cancer, liver cancer, intestinal cancer, esophageal cancer, breast cancer, cervical cancer, malignant lymphoma, nasopharyngeal cancer, or leukemia.

In a ninth aspect, the present disclosure provides a pharmaceutical composition.

According to an embodiment of the present disclosure, the pharmaceutical composition contains the above-mentioned recombinant vesicular stomatitis virus.

According to an embodiment of the present disclosure, the pharmaceutical composition is in a form suitable for inhalation or injection administration.

In a tenth aspect, the present disclosure provides a method for preventing or treating a tumor. According to an embodiment of the present disclosure, the method includes: administering, to a subject, at least one selected from the oncolytic virus according to the third or fifth aspect; or the pharmaceutical composition according to the ninth aspect. The method according to the embodiments of the present disclosure may enable the subject to have the ability of effectively preventing a tumor or inhibiting the growth of tumor cells.

According to an embodiment of the present disclosure, the method for preventing or treating the tumor may further include at least one of the following additional technical features.

According to an embodiment of the present disclosure, the tumor is selected from at least one of lung cancer, gastric cancer, liver cancer, intestinal cancer, esophageal cancer, breast cancer, cervical cancer, malignant lymphoma, nasopharyngeal cancer, or leukemia.

It should be understood that the above technical features of the present disclosure and the technical features specifically described below (e.g., in embodiments) may be combined with each other within the scope of the present disclosure, thereby constituting a new or preferred technical solution. Due to limited space, these technical features are not repeated one by one.

### BRIEF DESCRIPTION OF THE DRAWINGS

The above and/or additional aspects and advantages of the present disclosure will become obvious and easy to understand from the description of the embodiments in conjunction with the following accompanying drawings, in which:

FIG. 1 shows an analysis flowchart of human membrane receptor genes based on large samples of tumor tissues.

FIG. 2 shows jitter scatterplots of a proportion of patients whose corresponding receptor genes are significantly upregulated in each tumor.

FIG. 3 shows ZDOCK score results reflecting respective binding strengths of candidate ligands to tumor-specific receptors.

FIG. 4 shows a diagram of experimental results of screening ligands based on the screened receptors.

FIG. 5 shows expression quantities of mRNAs from CHRNA5, KISS1R, HTR1D, CCR8, and SSTR5 in BXPC3, HCT-8, HepG2, Su8686, H358, NCL-H460, and PANC1 cell samples detected by qPCR.

FIG. 6 shows killing effects of viruses, measured in cell killing assays, on BXPC3, HCT-8, HepG2, Su8686, H358, and PANC1 cells under different MOI conditions.

FIG. 7 shows killing effects of virus strains with different G proteins on NCL-H358 and NCL-H460 cells under different MOI conditions.

FIG. 8 shows killings effect of virus strains with inserted heterologous genes on NCL-H358 and NCL-H460 cells.

FIG. 9 shows a diagram of experimental results of an REV DQ408670.1 virus strain being safe to normal cells.

### DETAILED DESCRIPTION

Embodiments of the present disclosure are described in detail below. Examples of the embodiments are illustrated in the accompanying drawings. The embodiments described below with reference to the accompanying drawings are illustrative, and are intended to explain the present disclosure, rather than being construed as limitations to the present disclosure.

The present disclosure is accomplished on the basis of the inventors' following findings. The inventors have found that, when a coat surface of a vesicular stomatitis virus carries a special G protein, the G protein enables the vesicular stomatitis virus to have a high binding force to certain tumor cells through specific receptors on the surfaces of the tumor cells. Since the specific receptors on the surfaces of these tumor cells are expressed in a very low quantity in normal cells, the vesicular stomatitis viruses carrying such G proteins can be highly specific for the tumor cells. In addition, since the specific receptors on the surfaces of these tumor cells are highly expressed in a variety of tumor cells, the vesicular stomatitis viruses carrying such G proteins can improve the broad spectrum of tumor therapy, i.e., playing a killing function in the variety of tumor cells. Specifically, through extensive and in-depth researches and a large number of screenings, the inventors have first unexpectedly developed a genetically recombinant vesicular stomatitis virus for tumor therapy and its pharmaceutical composition. Specifically, in the recombinant VSV virus of the present disclosure, its glyco (G) protein may be at least one of a G protein with GenBank index number of X03633.1 and a G protein with GenBank index number of DQ408670.1, and a nuclear (N) protein, a phosphate (P) protein, matrix (M) protein, and an RNA-dependent RNA polymerase (L) thereof are derived from a Mudd summer virus strain. The experimental results indicate that the five tumor-specific receptors corresponding to the G protein are highly expressed in a variety of tumor cells, and the recombinant VSV virus of the present disclosure can bind to the five tumor cell-specific receptors to specifically infect the tumor cells. On this basis, the present disclosure is accomplished.

### Recombinant Vesicular Stomatitis Virus

In a first aspect, the present disclosure provides a recombinant vesicular stomatitis virus. According to an embodiment of the present disclosure, the recombinant vesicular stomatitis virus expresses a G protein. The G protein has an amino acid sequence set forth in SEQ ID NO: 1 or SEQ ID NO: 2, or an amino acid sequence having at least 80% homology to SEQ ID NO: 1 or 2.

(SEQ ID NO: 1)
MKCFLYLAFLFIGVNCKFTIVFPHNQKGNWKNVPSNYHYCPSSSD
LNWHNDLIGTGLQVKMPKSHKAIQADGWMCHASKWVTTCDFRWYG
PKYITHSIRSFTPSVEQCKESIEQTKQGTWLNPGFPPQSCGYATV
IDAEAVIVQVTPHHVLVDEYTGEWVDSQFINGKCSNDICPTVHNS
TTWHSDYKVKGLCDSNLISTDITFFSEDRELSSLGKEGTGFRSNY
FAYETGDKACKMQYCKHWGVRLPSGVWFEMADKDLFAAARFPECP
EGSSISAPSQTSVDVSLIQDVERILDYSLCQETWSKIRAGLPISP
VDLSYLAPKNPGTGPAFTIINGTLKYFETRYIRVDIAAPILSRMV
GMISGTTTERELWDDWAPYEDVEIGPNGVLRTSSGYKFPLYMIGH
GMLDSGLHLSSKAQVFEHPHIQDAASQLPDDEILFFGDTGLSKNP
IDFVEGWFSSWKSSIASFFFIIGLIIGLFLVLRVGIYLYIKLKHT
KKRQIYTDIEMNRLGR.
(SEQ ID NO: 2)
MKCLLYLAFLFIGVNCKFTIVFPHNQKGNWKNVPSNYHYCPSSSD
LNWHNDLIGTALQVKMPKSHKAIQADGWMCHASKWVTTCDFRWYG
PKYITHSIRSFTPSVEQCKESIEQTKQGTWLNPGFPPQSCGYATV
TDAEAVIVQVTPHHVLVDEYTGEWVDSQFINGKCSNYICPTVHNS
TTWHSDYKVKGLCDSNLISMDITFFSEDGELSSLGKEGTGFRSNY
FAYETGGKACKMQYCKHWGVRLPSGVWFEMADKDLFAAARFPECP
EGSSISAPSQTSVDVSLIQDVERILDYSLCQETWSKIRAGLPISP
VDLSYLAPKNPGTGPAFTIINGTLKYFETRYIRVDIAAPILSRMV
GMISGTTTERELWDDWAPYEDVEIGPNGVLRTSSGYKFPLYMIGH
GMLDSDLHLSSKAQVFEHPHIQDAASQLPDDESLFFGDTGLSKNP
IELVEGWFSSWKSSIASFFFIIGLIIGLFLVLRVGIHLCIKLKHT
KKRQIYTDIEMNRLGK.

According to an embodiment of the present disclosure, the recombinant vesicular stomatitis virus expressing the above G protein has stronger specific targeting ability to tumor cells, a broader spectrum of killing tumors, and a more significant killing effect.

It should be noted that the term “homology” described herein means that the amino acid sequences have similarity, and the differences of individual amino acids in the amino acid sequences do not affect the play of inherent protein functions. The term “homologous amino acid sequence” refers to an amino acid sequence derived from the substitution, deletion and addition of single or multiple amino acids in an amino acid sequence of a polypeptide. Specifically, the “having XXX sequence homology” described herein is calculated by the following formula:
1—number of differential amino acids/number of amino acids in a reference amino acid sequence*100%,
where the number of amino acids in the reference amino acid sequence refers to a number of amino acid sequences to be compared, for example, a reference amino acid sequence in the “G protein has at least 80% sequence homology to any of SEQ ID NO: 1 or SEQ ID NO: 2” is SEQ ID NO: 1 or SEQ ID NO: 2.

The above amino acid sequences having homology are biologically, chemically or structurally similar and have similar biological activities. “Structurally similar” refers to that amino acids have side chains of similar lengths, such as alanine, glycine, or serine, or side chains of similar sizes. “Chemically similar” means that amino acids have the same charges or are hydrophilic or hydrophobic. For example, hydrophobic residues, isoleucine, valine, leucine or methionine substitute each other. Alternatively, polar amino acids substitute each other. For example, lysine is substituted with arginine, aspartic acid is substituted with glutamic acid, asparagine is substituted with glutamine, threonine is substituted with serine, etc. “Biologically similar” means that amino acid sequences have sequence homology are similar in biological functions. For example, the recombinant VSV virus according to embodiments of the present disclosure has high affinity and binding force to broad spectrum and specificity of tumors.

The vesicular stomatitis viruses (VSV) are viruses belonging to vesicular viruses in the family of Rhabdoviridae and can be divided into two serotypes: New Jersey (VSV-NJ) and Indiana (VSV-IND). Virions are bullet-like or cylindrical virions, with a size of 150-180 nm×50-70 nm. The virus has an envelope, on which fibroids having a length of about 10 nm are uniformly and densely distributed. The viruses have a tightly-coiled spiral-symmetrical nucleocapsid inside. The viruses can be named according to classic vesicular lesions in the oral mucosa, dental pads, tongue, lips, nostrils, hooves and nipples of diseased animals, and transmitted by insect carriers, causing diseases that are only limited to their natural hosts, such as horses, cattle, and pigs. In human beings, the infections are mild and asymptomatic.

The VSV genome is an unsegmented single-stranded negative-stranded RNA (ssRNA) virus having a length of about 11 KB. From 3′-end to 5′-end, five non-overlapping genes N, NS, M, G, and L are arranged in sequence, which encode five different proteins, including nuclear (N) protein, phosphate (P) protein, matrix (M) protein, glyco(G)protein, and RNA-dependent RNA polymerase (L) protein, respectively. The 3′-end of the N gene is a Leader sequence, and the 5′-end of the N gene is a Trailor sequence, with a spacer sequence between the respective genes. A leader RNA at the 3′-end is the earliest virus transcript in infected cells, having a length of 47 nucleotides, it is neither capped nor translated, has functions that have not been clarified yet, and may inhibit the synthesis of host RNA. The N protein is essential to initiate genomic synthesis and can effectively protect virus RNA from digestion by various nucleases. The N protein has high immunogenicity, stimulates the body to produce cellular immunity, and plays an important role in transcription and replication. Therefore, the N protein may be essential to maintain an extended form of genomic RNA, and is related to replication and regulation. The P protein, VSV-NJ, has a homology of 41% to a VSV-IND virus strain, and functions to maintain virus's transcriptional activity together with a polymerase complex consisting of polymerase L and nuclear protein N, and genomic RNA. The M protein plays a key role in virus pathogenesis and virus replication, is rich in basic amino acids and contains a highly basic amino-terminal domain. The M protein can inhibit transcription by binding to a nucleocapsid, while assisting virus germination from the host, and is the only polypeptide involved in the germination process. The G protein is a main surface antigen of a virus, determines the virulence of the virus, and is also a protective antigen of the virus. The G protein may stimulate the body to produce neutralizing antibodies. The L gene encodes an RNA poly E protein, which may determine the transcriptional activity of RNA, and bind to the P protein to catalyze the replication of mRNA. This protein is a core component of a polymerase complex and a replicase complex, and involves initiation, extension, methylation, capping, poly(A) tail formation, and the like. In addition, there is an extensive homology in spacer sequences between each gene, and these sequences have a common structure, namely 3′-AUAC(U) 7NAUUGUCNN-UAG-5′. A conserved sequence between these genes is a key signal to affect the activity of a polymerase or the cleavage activity of an enzyme, whereas during replication, these signals are masked and do not work.

The present disclosure provides a recombinant vesicular stomatitis virus (VSV) virus. As used herein, the terms “recombinant VSV virus”, “recombinant vesicular stomatitis virus”, “recombinant virus of the present disclosure” are used interchangeably, referring to the recombinant VSV virus capable of specifically infecting tumor cells, as described in the first aspect of the present disclosure. The recombinant VSV virus specifically infects tumor cells, and specifically binds to specific receptors selected from CHRNA5, SSTR5, KISS1R, HTR1D, or CCR of the tumor cells.

According to an embodiment of the present disclosure, the G protein includes an amino acid sequence having at least 90%, at least 95%, at least 98%, or at least 99% homology to (a) or (b).

According to an embodiment of the present disclosure, the recombinant vesicular stomatitis virus carries no heterologous gene. The inventors have found that the recombinant vesicular stomatitis virus carrying no heterologous gene has a significantly higher killing effect on tumor cells than a recombinant vesicular stomatitis virus carrying a foreign gene. According to an embodiment of the present disclosure, the term “heterologous gene” described herein refers to a gene that has not been reported in wild-type vesicular stomatitis viruses, unless otherwise specified. Alternatively, in other words, all encoded proteins in the recombinant vesicular stomatitis viruses are expressed in the wild-type vesicular stomatitis viruses.

According to an embodiment of the present disclosure, a gene coding sequence of a genome of the recombinant vesicular stomatitis virus is selected from wild-type vesicular stomatitis viruses.

According to an embodiment of the present disclosure, the recombinant vesicular stomatitis virus is allowed to carry a nucleic acid molecule encoding the G protein.

According to an embodiment of the present disclosure, the virus protein has a ZDOCK score of a binding force to a cell receptor not lower than 1,800. Those skilled in the art can understand that, by entering sequences of the G protein and the cell receptor, a binding force characterization parameter “ZDOCK score” between the G protein and the cell receptor may be easily obtained. The inventors have found that when the ZDOCK score is 1,800, e.g., not lower than 1,900, not lower than 2,000, preferably not lower than 2,100, a binding force of a virus carrying the G protein to a tumor cell carrying the corresponding receptor can be significantly improved. According to an embodiment of the present disclosure, the ZDOCK score may be determined by conventional software (for example, see Pierce B G, Hourai Y, Weng Z. (2011) Accelerating Protein Docking in ZDOCK Using an Advanced 3D Convolution Library. PLOS One 6(9): e24657).

According to an embodiment of the present disclosure, the G protein includes at least one of a G protein with GenBank index number of X03633.1 and a G protein with GenBank index number of DQ408670.1. The inventors of the present disclosure have unexpectedly found that the G protein with GenBank index number of X03633.1 and the G protein with GenBank index number of DQ408670.1 have a significantly stronger binding force to a receptor for tumor cells than other G proteins.

According to an embodiment of the present disclosure, the recombinant vesicular stomatitis virus further expresses at least one selected from a nuclear protein, a phosphate protein, a matrix protein, and an RNA-dependent RNA polymerase. The inventors have unexpectedly found that the recombinant virus, which is constructed by combining these proteins with at least one of the G protein with GenBank index number of X03633.1 and the G protein with GenBank index number of DQ408670.1, has stronger tumor killing activity. In the inventors' opinion, the mechanism thereof may be in that for tumor cells, a variety of protein combinations from different sources may trigger immune responses other than those of protein combinations from the same source, thereby further improving the killing effect on the tumor cells.

Correspondingly, the recombinant vesicular stomatitis virus carries a nucleic acid molecule encoding the nuclear protein, a nucleic acid molecule encoding the phosphate protein, a nucleic acid molecule encoding the matrix protein, or a nucleic acid molecule encoding the RNA-dependent RNA polymerase.

Preferably, at least one of the nucleic acids that encode the nuclear protein, the phosphate protein, the matrix protein, and the RNA-dependent RNA polymerase is derived from a Mudd summer subtype virus strain of the vesicular stomatitis virus. The inventors have unexpectedly found that the recombinant virus which is constructed by combining these proteins with at least one of the G protein with GenBank index number of X03633.1 and the G protein with GenBank index number of DQ408670.1 has stronger tumor killing activity. In the inventors' opinion, the mechanism thereof may be in that for tumor cells, a variety of protein combinations from different sources may trigger immune responses other than those of protein combinations from the same source, thereby further improving the killing effect on the tumor cells.

### Methods for Constructing Recombinant Vesicular Stomatitis Virus

In a second aspect, the present disclosure provides a method for constructing a recombinant vesicular stomatitis virus. According to an embodiment of the present disclosure, the method includes: allowing an initial vesicular stomatitis virus to express a G protein. The G protein has the following amino acid sequence: (a) SEQ ID NO: 1; (b) SEQ ID NO: 2; or (c) an amino acid sequence having at least 80% homology to (a) or (b). Optionally, the G protein is derived from wild-type vesicular stomatitis viruses. Optionally, the G protein includes at least one of a G protein with GenBank index number of X03633.1 and a G protein with GenBank index number of DQ408670.1. Optionally, the G protein has an amino acid sequence having at least 90%, at least 95%, at least 98%, or at least 99% or at least 99% homology to (a) and (b). As described above, according to an embodiment of the present disclosure, the recombinant vesicular stomatitis virus expressing the above G protein has stronger specific targeting ability to tumor cells, a broader spectrum of killing tumors, and a more significant killing effect.

According to an embodiment of the present disclosure, the initial oncolytic stomatitis virus further expresses at least one selected from a nuclear protein, a phosphate protein, a matrix protein, and an RNA-dependent RNA polymerase. The inventors have unexpectedly found that the recombinant virus, which is constructed by combining these proteins with at least one of the G protein with GenBank index number of X03633.1 and the G protein with GenBank index number of DQ408670.1, has stronger tumor killing activity. In the inventors' opinion, the mechanism thereof may be in that for tumor cells, a variety of protein combinations from different sources may trigger immune responses other than those of protein combinations from the same source, thereby further improving the killing effect on the tumor cells. These nuclear proteins, phosphate proteins, matrix proteins, and RNA-dependent RNA polymerases are not limited, because the inventors have found that different nuclear proteins, phosphate proteins, matrix proteins, and RNA-dependent RNA polymerases can achieve good technical effects once the specific G protein is selected.

According to an embodiment of the present disclosure, the initial oncolytic stomatitis virus is allowed to carry: a nucleic acid molecule encoding the nuclear protein, a nucleic acid molecule encoding the phosphate protein, a nucleic acid molecule encoding the matrix protein, or a nucleic acid molecule encoding the RNA-dependent RNA polymerase. Optionally, at least one of the nucleic acids that encode the nuclear protein, the phosphate protein, the matrix protein, and the RNA-dependent RNA polymerase is derived from an exemplary virus strain, e.g., a Mudd summer subtype virus strain of the vesicular stomatitis virus. Therefore, the expression efficiency of these proteins and the killing effect of the recombinant oncolytic virus on tumors may be further improved.

### Method for Improving Binding Force of Vesicular Stomatitis Virus to Target Cell

In a third aspect, the present disclosure provides a method for improving a binding force of a vesicular stomatitis virus to a target cell. According to an embodiment of the present disclosure, the method includes: allowing the vesicular stomatitis virus to express a G protein, the G protein including the following amino acid sequence: (a) SEQ ID NO: 1; (b) SEQ ID NO: 2; or (c) an amino acid sequence having at least 80% homology to (a) or (b). As described above, according to an embodiment of the present disclosure, by expressing the G protein described above, the binding force of the virus binding to the target cell through a cell surface receptor can be effectively enhanced.

According to an embodiment of the present disclosure, the G protein is derived from wild-type vesicular stomatitis viruses.

According to an embodiment of the present disclosure, the G protein includes at least one of a G protein with GenBank index number of X03633.1 and a G protein with GenBank index number of DQ408670.1.

According to an embodiment of the present disclosure, the G protein comprises an amino acid sequence having at least 90%, at least 95%, at least 98%, or at least 99% homology to (a) or (b).

According to an embodiment of the present disclosure, the initial vesicular stomatitis virus is allowed to carry a nucleic acid molecule encoding the G protein.

### Pharmaceutical Composition and its Pharmaceutical Use

In a fourth aspect, the present disclosure provides a pharmaceutical composition. According to an embodiment of the present disclosure, the pharmaceutical composition contains the recombinant vesicular stomatitis virus described above.

According to an embodiment of the present disclosure, the pharmaceutical composition is in a form suitable for inhalation or injection administration.

Furthermore, the present disclosure provides use of the above-mentioned pharmaceutical composition or recombinant vesicular stomatitis virus in the preparation of a medicament, and the medicament is for use in the treatment or prevention of a cancer or a tumor.

According to an embodiment of the present disclosure, the cancer is selected from at least one of lung cancer, gastric cancer, liver cancer, intestinal cancer, esophageal cancer, breast cancer, cervical cancer, malignant lymphoma, nasopharyngeal cancer, or leukemia.

The pharmaceutical composition provided by the present disclosure includes a pharmaceutically acceptable carrier and an effective amount of the following active ingredient: the recombinant VSV virus of the present disclosure that specifically infects tumor cells.

As used herein, the term “effective amount” or “effective dose” refers to an amount that produces a function or activity on human beings and/or animals and is acceptable to human beings and/or animals.

As used herein, a “pharmaceutically acceptable” ingredient is a substance that is appropriate for human beings and/or mammals without excessive adverse side effects (e.g., toxicity, irritation and allergy), i.e., a substance with a reasonable benefit/risk ratio. The term “pharmaceutically acceptable carrier” refers to a carrier used for therapeutic agent administration, including various excipients and diluents.

The pharmaceutical composition of the present disclosure includes a safe and effective amount of the active ingredient of the present disclosure and a pharmaceutically acceptable carrier. Such carrier includes, but not limited to: saline, buffer, glucose, water, glycerin, ethanol, or combinations thereof. A pharmaceutical preparation should generally match a route of administration. Formulations of the pharmaceutical composition of the present disclosure include an injection, an oral preparation (tablets, capsules, or oral liquid), a transdermal agent, and a sustained release agent. For example, the pharmaceutical composition is prepared from normal saline or an aqueous solution containing glucose and other adjuvants by means of conventional methods. The pharmaceutical composition should be manufactured under sterile conditions.

In an embodiment, the pharmaceutical composition of the present disclosure may further include an additional tumor therapeutic agent.

The effective amount of the active ingredient described in the present disclosure may vary with the route of administration and the severity of a disease to be treated. The selection of the preferred effective amount may be determined by those of ordinary skill in the art according to various factors (e.g., through clinical tests). The factors include, but are not limited to: pharmacokinetic parameters of the active ingredient such as bioavailability, metabolism, and half-life; and the severity of the disease to be treated, a weight of a patient, an immune status of the patient, the route of administration, etc. For example, depending on the exigencies of treatment conditions, several divided doses may be given per day, or the dose may be reduced proportionally.

The pharmaceutically acceptable carrier described in the present disclosure includes, but not limited to, water, saline, liposomes, lipids, proteins, protein-antibody conjugates, peptide substances, celluloses, nanogels, or combinations thereof. The selection of the carrier should match the route of administration, which is well known to those of ordinary skill in the art.

The present disclosure has the following main advantages.

1) The G protein found in the present disclosure has a higher binding force to tumor-specific receptors than other general G proteins, thereby ensuring that the G protein has strong selectivity for tumor cells.

2) The recombinant VSV virus of the present disclosure has high specificity for tumor cells, and has almost no effect on normal tissues and cells, thereby achieving better safety performances and fewer side effects.

3) The experimental results indicate that the recombinant VSV virus of the present disclosure has significant oncolytic and killing effects on a variety of tumor tissues and cells, thereby achieving high killing efficiency and good targeting ability.

The present disclosure is described below with reference to specific examples. It should be noted that these examples are only descriptive, rather than limiting the present disclosure in any way.

### Example 1: Analysis of Human Membrane Receptor Genes Based on Large Samples of Tumor Tissues

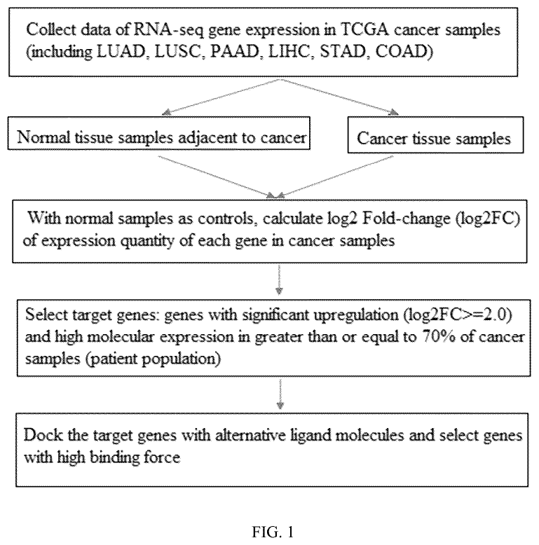

A method for analysis of human membrane receptor genes based on large samples of tumor tissues is described in detail below with reference to FIG. 1.

### 1.1 Preprocessing and Analysis of Human Membrane Receptor Genes and Expression Data Thereof

In the present disclosure, receptor gene information expressed in human cells has been summarized from existing studies (Reference: (Synchronous birth is a dominant pattern in receptor-ligand evolution, BMC Genomics. Grandchamp and Monget, 2018 Aug. 14; 19(1): 611). The inventors have downloaded a gene expression matrix (normalized value), gene mutation information, and related clinical data of cancer patients from UCSC Xena (http://xena.ucsc.edu/). Cancers included in the data are: adrenocortical carcinoma, bladder urothelium carcinoma, breast invasive carcinoma, cervical squamous cell carcinoma & adenocarcinoma, cholangiocarcinoma, colon adenocarcinoma, colon endocervical adenocarcinoma/adenocarcinoma of esophagus, lymphoid neoplasms & diffuse large B-cell lymphoma, esophageal cancer, FFPE test stage II, glioblastoma, glioma, head & neck squamous cell carcinoma, renal chromosome, pan-renal cohort (KICH+KIRC+KIRP), renal clear cell carcinoma, renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma & paraganglioma, prostate adenocarcinoma, rectum adenocarcinoma, sarcoma, cutaneous melanoma, gastric adenocarcinoma, gastric and esophageal cancer, germinal cell tumor of testis, thyroid carcinoma, thymoma, uterine corpus endometrioid carcinoma, uterine carcinosarcoma, and uveal melanoma.

The inventors first removed less than three sample tumors and normal tissue information from the downloaded data, and then performed differential expression analysis. The inventors employed Limma software (version: 3.38.3) to perform differential expression analysis (Reference: (Limma Powers Differential Expression Analyses for RNA-Sequencing and Microarray Studies. Nucleic Acids Research, 43, e47, Ritchie, M. E., et al. (2015). A voom model of a limma R package is used in the analysis. Only when a gene meets a criterion |log 2FC|>1, the gene having P-value<0.05 will be recognized as a differential gene.

### 1.2 Data Analysis

P-values and a fold (log 2FC) of difference in gene expression of membrane receptors between groups were calculated using an R language. |log 2FC| being selected to be greater than or equal to 2.0 was considered to have significant upregulation/downregulation of differentially expressed genes. A p-value of t-test less than 0.01 was determined to be statistically significant. A ComplexHeatmap R package was used to generate a heatmap of a log 2FC matrix for each comparative group pair.

Then, according to a series of screening conditions, genes (i.e., genes with |log 2FC|>2.0) significantly upregulated in more than or equal to 70% of cancer samples in colorectal cancer, lung cancer, pancreatic cancer, gastric cancer and liver cancer species were selected; and more than 10 receptors were selected under conditions such as high background expression.

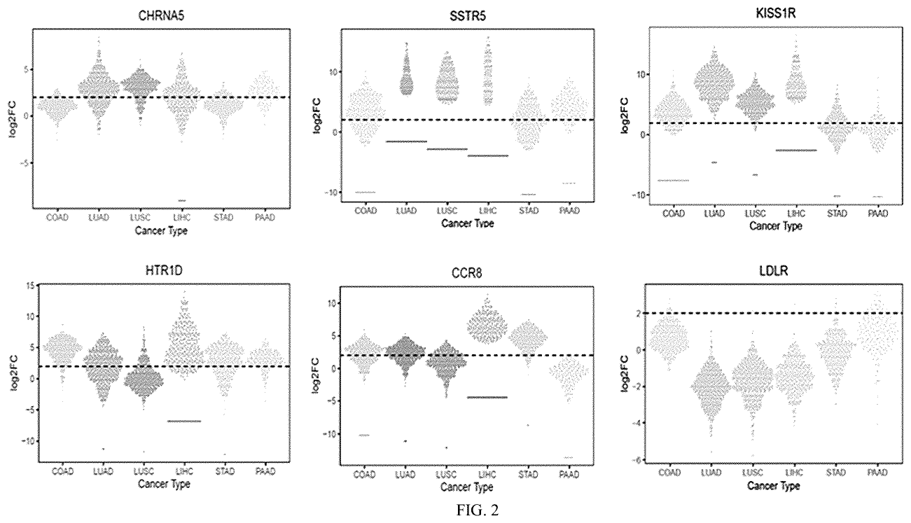

Specifically, a jitter scatter plot (as shown in FIG. 2) of log 2FC values for each gene in different tumor samples was drawn using ggplot2 and ggbeeswarm software, in order to present a proportion of patients in which this gene was significantly upregulated in each tumor.

In addition, 13 screened receptors were molecularly docked with candidate ligands, and 5 receptors with optimal binding force were finally selected.

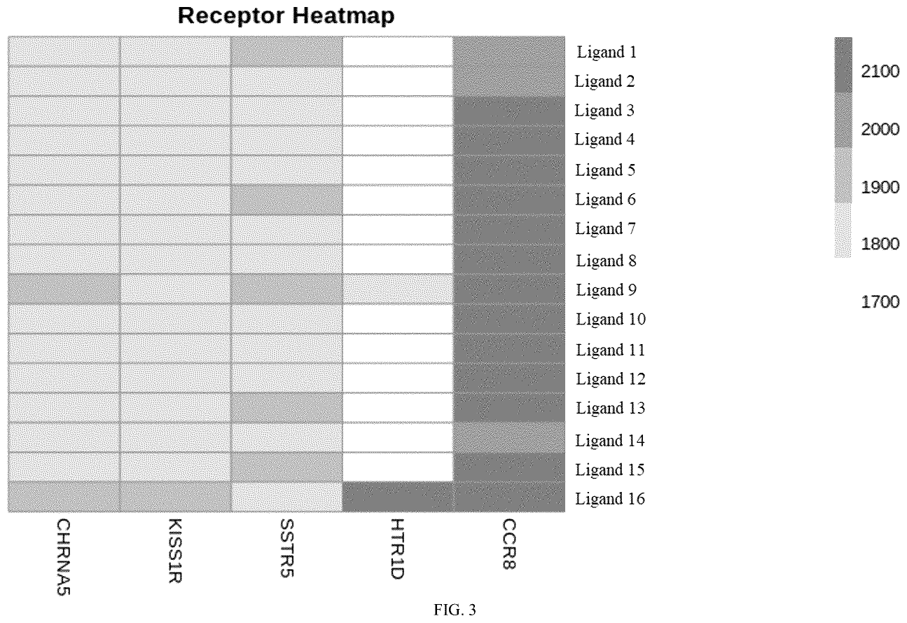

The results are shown in FIG. 3. The serial numbers of ligands shown in FIG. 3, the corresponding names of ligands, and amino acid sequence capture numbers are listed in Table 1. Nicotinic choline receptor α5 (CHRNA5), somatostatin receptor 5 (SSTR5), kisspeptin receptor (KISS1R), serotonin receptor 1D (HTR1D), and C—C chemokine receptor 8 (CCR8) are receptor proteins differentially expressed in tumor cells and normal cells.

TABLE 1

| Serial | | Amino acid sequence |
|---|---|---|
| number of | | capture number |
| ligands | Name of ligands | (GENE ID) |
| 1 | M35219.1-lig-FL | M35219.1 |
| 2 | HQ593628.1-lig-FL | HQ593628.1 |
| 3 | KP872888.1-lig-FL | KP872888.1 |
| 4 | HQ588112.1-lig-FL | HQ588112.1 |
| 5 | MH919398.1-lig-FL | MH919398.1 |
| 6 | MK934319.1-lig-FL | MK934319.1 |
| 7 | NC-038236.1- lig-FL | NC038236.1 |
| 8 | EU849003.1-lig-FL | EU849003.1 |
| 9 | X03633.1-lig-FL | X03633.1 |
| 10 | EF197793.1-X259G-lig-FL | EF197793.1 |
| 11 | M35214.1-lig-FL | M35214.1 |
| 12 | KF880910.1-lig-FL | KF880910.1 |
| 13 | AF473865.1-lig-FL | AF473865.1 |
| 14 | KU296059.1-X128G-lig-FL | KU296059.1 |
| 15 | MH919397.1-lig-FL | MH919397.1 |
| 16 | DQ408670.1-lig-FL | DQ408670.1 |

### Example 2: Selection of Virus Ligands Based on Receptors

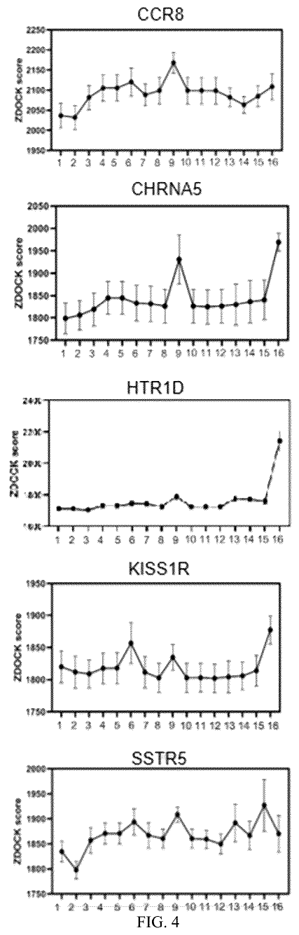

The inventors selected 16 homologous ligands of the vesicular stomatitis virus, which were then modeled and docked with 5 tumor-specific receptors screened in Example 1. The obtained results of docking were ranked based on ZDOCK scores. The binding is stronger and the confidence level in the results is higher with an increased in the score. At the same time, through comprehensive analysis of clustering results of these conformations, it was found that the ZDOCK scores are shape complementarity scores calculated by a ZDOCK program, and based on parameter settings, the ZDOCK scores may further include electrostatic and solvent removal energy terms. It is better when the ZDOCK score is higher. Furthermore, the inventors evaluated the binding strength by means of a ZDOCK score function, and screened out the ligands having strong binding ability to tumor-specific receptors (the results are shown in FIG. 4). The ligands with the optimal binding effect are DQ408670.1-lig-F and X03633.1-lig-FL, and the corresponding amino acid sequence capture numbers thereof are DQ408670.1 and GENE ID: X03633.1, respectively.

### Example 3: Construction and Amplification of Recombinant Vesicular Stomatitis Virus Based on Different Serotype Proteins

The inventors constructed recombinant vesicular stomatitis viruses REV DQ408670.1, REV X03633.1, REV KP872888.1, and REV HQ593628.1 by combining a combination of L, N, P, and M proteins derived from the Mudd summer subtype virus strain with a G protein having a capture sequence number of GENE ID: DQ408670.1, GENE ID: X03633.1, GENE ID: KP872888.1, or GENE ID: HQ593628.1, respectively,

A packaging method for the virus strains REV DQ408670.1, REV X03633.1, REV KP872888.1, and REV HQ593628.1 is described below.

In vitro recombination of VSV requires: a full-length plasmid containing a virus genome (containing the G protein), and helper plasmids for a skeleton protein required (N, P, L, M) for virus packaging. The plasmid was transferred into BHK21 cells by means of in vitro transfection, and the virus was assembled and matured inside the cells, germinated, and then released out of the cells (Reference: Vesicular stomatitis virus-based vaccine protects hamsters against lethal challenge with Andes virus. Journal of virology 85, 12781-12791, doi:10.1128/JVI.00794-11 (2011), Brown, K. S., Safronetz, D., Marzi, A., Ebihara, H. & Feldmann, H.).

Vero cells were used for the expansion of the virus. A certain titer of virus was added to the cultured Vero cells. The virus may infect the cells, and may self-replicate in the cells. The mature viruses were released into a supernatant of cell culture, and the supernatant of cell culture was concentrated to obtain a virus concentrate. The titer was determined for subsequent experiments.

### Example 4: Detection of Tumor Cell Receptors and Cell Killing Effects

In the present example, different viruses constructed in Example 3 are used to verify the killing effects on different tumor cells.

4.1 q-PCR test:

1×10⁶ of BXPC3, HCT-8, HepG2, Su8686, H358, NCL-H460 (H460) and PANC1 cell samples were extracted by means of a Trizol method, reverse transcription of a 20 μL system was performed with 500 ng/μL of RNA, and mRNA expressions of CHRNA5, KISS1R, HTR1D, CCR8 and SSTR5 genes were detected in 7 cell samples by means of fluorescent quantitative PCR by a SYBR GREEN method.

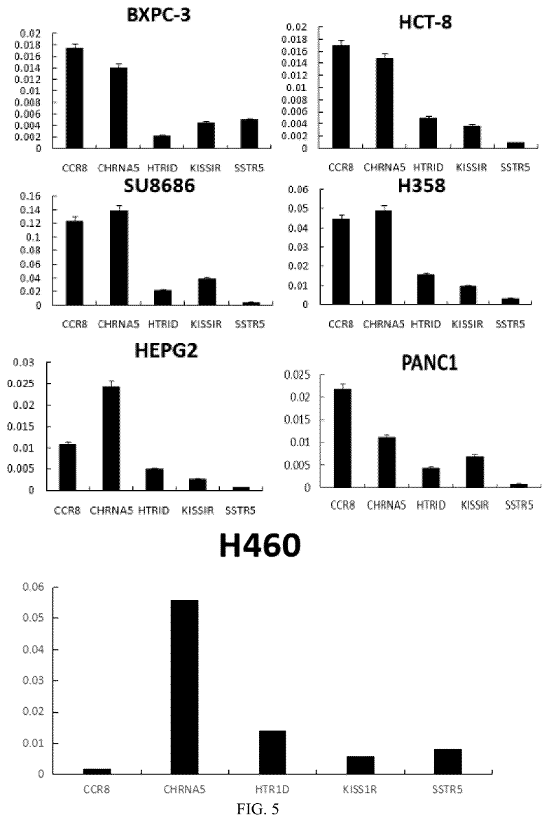

The results are shown in FIG. 5. The qPCR results indicate that the mRNA expression levels of CHRNA5 receptor genes in BXPC3, HCT-8, HepG2, Su8686, H358, NCL-H460, and PANC1 cell samples were relatively high, but there were differences in receptors with high relative expression quantities in different cells. For example, CHRNA5 and HTR1D receptors had highest expression quantities in H460 cells, while the gene expression levels of CHRNA5 and CCR8 receptors were relatively high in other cells.

### 4.2 Cell Killing Experiment (CCK):

BXPC3, HCT-8, HepG2, Su8686, H358, and PANC1 cells in good condition were prepared into cell suspension (5×10⁴ cell/mL), and the cell suspension was added to a 96-well plate at 100 μL/well, and a medium was filled to reduce evaporation. The cells were cultured overnight. The virus with a known titer was diluted into virus working solutions of MOI: 0.01, MOI: 0.1 and MOI: 1 using Opti-MEM. The culture solution in the 96-well plate was discarded by suction. 50 μL of virus diluent was added per well, with repeated triplication for each diluent. The Opti-MEM, as a blank control, was additionally added into wells for repeated triplication. The solution was changed 2 hours after the virus diluent was added, by adding 100 μL of 1% FBS medium per well. After 48/72 h, 10 μL of CCK8 test solution was added per well, and incubated at 37° C. for 2 h, and readings were obtained by reading with a OD450 microplate reader.

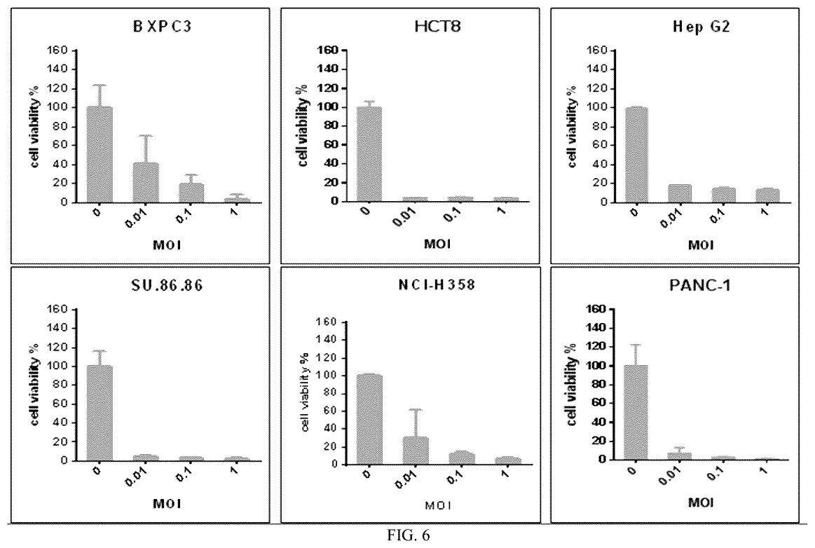

FIG. 6 shows CCK killing effects of REV DQ408670.1 on different cells. The CCK detection results indicate that the virus working solutions of REV DQ408670.1 in MOI: 0.01, MOI: 0.1 and MOI: 1 all have significant killing effects on BXPC3, HCT-8, HepG2, Su8686, H358 and PANC1 cells.

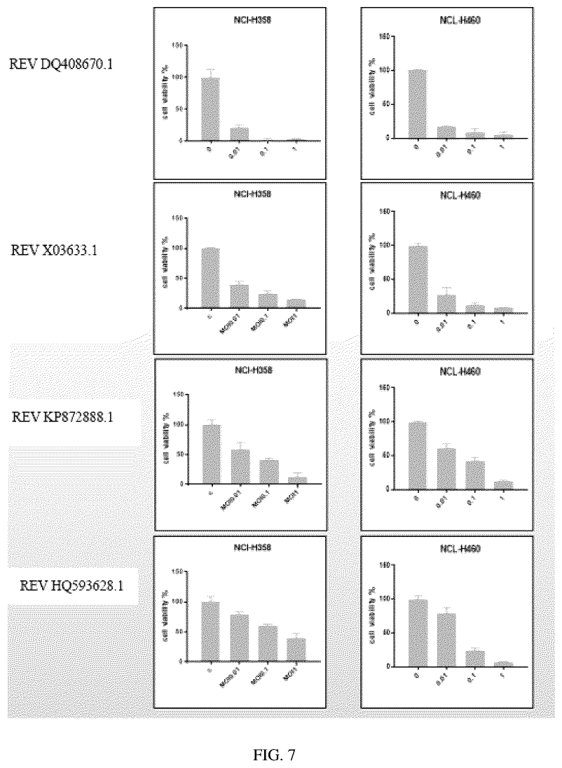

The experimental results are shown in FIG. 7. The CCK results indicate that the killing effects of the virus working solutions of REV DQ408670.1 and REV X03633.1 in MOI: 0.01, MOI: 0.1 and MOI: 1 on NCL-H358 and NCL-H460 cells are significantly better than those of REV KP872888.1 and REV HQ593628.1. At the same time, in combination with the results in FIG. 5, the expression quantities of CCR8 and CHRNA5 in NCL-H358 are relatively high, and the expression quantities of CHRNA5 and HTR1D in NCL-H460 are relatively high. In addition, in combination with binding heatmap results of the binding of the receptors to ligands in FIG. 3, G proteins of DQ408670.1 and X03633.1 have strong binding forces to CCR8 and HTR1D receptors. In summary, these results reveal that when the recombinant vesicular stomatitis virus has a high binding force to the tumor cell receptor, the killing effect of the recombinant virus on tumor cells that highly express the receptor is more significant.

### Example 5: Killing Effects of Virus Strains with Different Combinations of L, N, P, and M on Tumor Cells, Based on Selected G Protein

### Cell Killing Experiment (CCK):

Using an antisense genetics method, the inventors constructed a virus strain REV DQ408670.1, a virus strain REV DQ408670.1-V1, and a virus strain REV DQ408670.1-V2. The REV DQ408670.1-V1 is a virus strain based on the REV DQ408670.1 virus strain by changing L and M proteins, and REV DQ408670.1-V2 is a virus strain based on the REV DQ408670.1 virus strain by changing N and P proteins.

5×10⁴ cell/mL of cell suspension, which was prepared from H358 and H460 cells in good condition, was added into a 96-well plate at 100 μL/well, the medium was filled to reduce the evaporation, and the cell suspension is cultured overnight. Three virus strains with known titers are diluted into a virus working solution of MOI: 0.01 using Opti-MEM. The culture solution in the 96-well plate was discarded by suction. 50 μL of virus diluent was added per well, with repeated triplication for each diluent. The Opti-MEM, as a blank control, was additionally added for repeated triplication. The solution was changed 2 hours after the virus diluent was added, by adding 100 μL of 1% FBS medium per well. After 72 h, 10 μL of CCK8 test solution was added per well, and incubated at 37° C. for 2 h, and readings were obtained by reading with a OD450 microplate reader.

The experimental results are shown in Table 2. The results of the three virus strains REV DQ408670.1, REV DQ408670.1-V1 and REV DQ408670.1-V2 are similar, and the virus working solution of MOI: 0.01 has significant killing effects on H358 and H460 cells.

TABLE 2
Inhibition rates (%) of virus on tumor cells at MOI: 0.01

| | REV | REV | REV |
|---|---|---|---|
| | DQ408670.1 | DQ408670.1-V1 | DQ408670.1-V2 |
| H358 | 88.07 | 78.84 | 85.27 |
| H460 | 83.33 | 81.33 | 79.36 |

### Example 6: Tumor Cells Killing Effects of Virus Strain Based on Selected G Protein and Inserted with a Foreign Gene

### Cell Killing Experiment (CCK):

Using an antisense genetics method, the inventors constructed a virus strain FJ-INFβ by inserting a heterologous gene INFβ in the constructed virus strain REV DQ408670.1.

5×10⁴ cell/mL of cell suspension, which was prepared from H358 and H460 cells in good condition, was added into a 96-well plate at 100 μL/well, the medium was filled to reduce the evaporation, and the cell suspension was cultured overnight. The virus strains REV DQ408670.1 and FJ-INFβ having known titers were diluted into virus working solutions of MOI: 0.01, MOI: 0.1 and MOI: 1 using Opti-MEM, respectively. The culture solution in the 96-well plate was discarded by suction. 50 μL of virus diluent was added per well, with repeated triplication for each diluent. The Opti-MEM, as a blank control, was additionally added or repeated triplication. The solution was changed 2 hours after the virus diluent was added, by adding 100 μL of 1% FBS medium per well. After 72 h, 10 μL of CCK8 test solution was added per well, and incubated at 37° C. for 2 h, and readings were obtained by reading with a OD450 microplate reader.

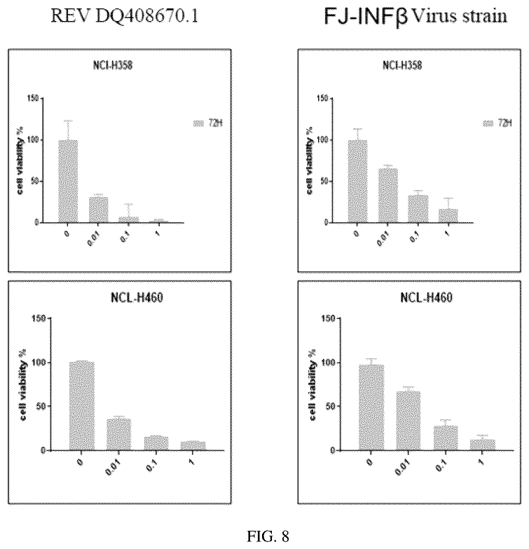

The results are shown in FIG. 8. The virus strain REV DQ408670.1 has significantly better killing effects on H358 and H460 cells than the virus strain FJ-INFβ.

### Example 7: Detection of Normal Cell Killing Effect of Virus Strain REV DQ408670.1

### Cell Killing Experiment (CCK):

5×10⁴ cell/mL of cell suspension, which was prepared from lung normal cells BEAS-2B in good condition, was added into a 96-well plate at 100 μL/well, the medium was filled to reduce the evaporation, and the cell suspension was cultured overnight. A virus REV DQ408670.1 with a known titer was diluted into virus working solutions of MOI: 0.01, MOI: 0.1 and MOI: 1 using Opti-MEM. The culture solution in the 96-well plate was discarded by suction. 50 μL of virus diluent was added per well, with repeated triplication for each diluent. The Opti-MEM, as a blank control, was additionally added for repeated triplication. The solution was changed 2 hours after the virus diluent was added, by adding 100 μL of 1% FBS medium per well. After 72 h, 10 μL of CCK8 test solution was added per well, and incubated at 37° C. for 2 h. Readings were obtained by reading with a OD450 microplate reader.

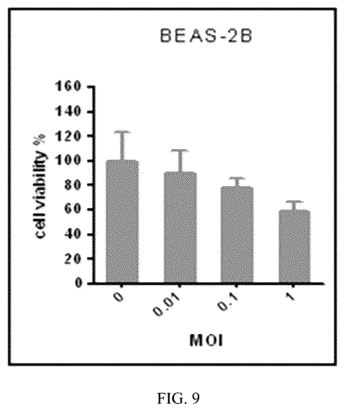

The experimental results are shown in FIG. 9. The CCK test results indicate that all the working solutions of the virus strain REV DQ408670.1 at MOI: 0.01, MOI: 0.1 and MOI: 1 have no obvious killing effect on BEAS-2B cells.

In the present disclosure, the description of referring terms such as “an embodiment”, “some embodiments”, “an example”, “a specific example” and “some examples” means that particular features, structures, materials or characteristics described in combination with the embodiment(s) or example(s) are included in at least one embodiment or example of the present disclosure. In the present disclosure, schematic description of the above terms does not necessarily refer to the same embodiment or example. Furthermore, the described particular features, structures, materials or characteristics can be integrated with any one or more embodiments or examples in a suitable manner. In addition, in the absence of contradiction, those skilled in the art can integrate and combine different embodiments or examples described in this specification and the features of different embodiments or examples.

Although the embodiments of the present disclosure have been illustrated and described above, it can be understood that the above embodiments are illustrative and should not be construed as limitations of the present disclosure. Those skilled in the art can make

1.-10. (canceled)

11. A method for constructing a recombinant oncolytic virus, comprising: allowing an initial vesicular stomatitis virus to express a G protein, the G protein comprising the following amino acid sequence:
(a) SEQ ID NO: 1;
(b) SEQ ID NO: 2; or
(c) an amino acid sequence having at least 80% homology to (a) or (b),
optionally, the G protein is derived from wild-type vesicular stomatitis viruses, and
optionally, the amino acid sequence of the G protein has a capture sequence number of GENE ID: DQ408670.1 or GENE ID: X03633.1; and
optionally, the amino acid sequence of the G protein has at least 90%, at least 95%, at least 98%, or at least 99% homology to (a) or (b).

12. The method according to claim 11, wherein the initial vesicular stomatitis virus further expresses at least one selected from a nuclear protein, a phosphate protein, a matrix protein, and an RNA-dependent RNA polymerase.

13. The method according to claim 12, wherein the initial vesicular oncolytic virus carries: a nucleic acid molecule encoding the nuclear protein, a nucleic acid molecule encoding the phosphate protein, and a nucleic acid molecule encoding the matrix protein; and
optionally, at least one of the nucleic acid molecule encoding the nuclear protein, the nucleic acid molecule encoding the phosphate protein, the nucleic acid molecule encoding the matrix protein, and a nucleic acid molecule encoding the RNA-dependent RNA polymerase is derived from a Mudd summer subtype virus strain of the vesicular stomatitis virus.

14. A recombinant oncolytic virus, being constructed by the method according to claim 11.

15. A method for improving a binding force of an oncolytic virus to a target cell, the method comprising:
allowing the oncolytic virus to express a G protein having a strong binding force to the target cell, the G protein including the following amino acid sequence:
(a) SEQ ID NO: 1;
(b) SEQ ID NO: 2; or
(c) an amino acid sequence having at least 80% homology to (a) or (b),
optionally, the G protein is derived from wild-type vesicular stomatitis viruses, and optionally, the amino acid sequence of the G protein has a capture sequence number of GENE ID: DQ408670.1 or GENE ID: X03633.1; and
optionally, the G protein comprises an amino acid sequence having at least 90%, at least 95%, at least 98%, or at least 99% homology to (a) or (b).

16. A recombinant oncolytic virus obtained by the method according to claim 15.

17. A recombinant oncolytic virus having a high affinity for a tumor cell, wherein the recombinant oncolytic virus expresses a virus protein having a high affinity for a cell receptor, the virus protein being selected from:
(a) SEQ ID NO: 1;
(b) SEQ ID NO: 2; or
(c) an amino acid sequence having at least 80% homology to (a) or (b).

18. The recombinant oncolytic virus according to claim 17, wherein the virus protein is derived from wild-type vesicular stomatitis viruses.

19. The recombinant oncolytic virus according to claim 18, wherein the virus protein comprises an amino acid sequence having at least 90%, at least 95%, at least 98%, or at least 99% homology to (a) or (b).

20. The recombinant oncolytic virus according to claim 17, wherein the recombinant oncolytic virus carries no heterologous gene.

21. The recombinant oncolytic virus according to claim 17, wherein each gene coding sequence of a genome of the recombinant oncolytic virus is selected from wild-type vesicular stomatitis viruses.

22. The recombinant oncolytic virus according to claim 17, wherein the virus protein has a ZDOCK score of a binding force to a cell receptor not lower than 1,800.

23. The recombinant oncolytic viruses according to claim 17, wherein the cell receptor comprises at least one selected from CHRNA5, SSTR5, KISS1R, HTR1D, CCR8.

24. The recombinant oncolytic virus according to claim 17, wherein the recombinant oncolytic virus further expresses at least one selected from a nuclear protein, a phosphate protein, a matrix protein, and an RNA-dependent RNA polymerase.

25. The recombinant oncolytic virus according to claim 17, wherein the genome of the recombinant oncolytic virus carries: a nucleic acid molecule encoding the nuclear protein, a nucleic acid molecule encoding the phosphate protein, a nucleic acid molecule encoding the matrix protein, and a nucleic acid molecule encoding the RNA-dependent RNA polymerase.

26. The recombinant oncolytic virus according to claim 25, wherein at least one of the nucleic acid molecule encoding the nuclear protein, the nucleic acid molecule encoding the phosphate protein, the nucleic acid molecule encoding the matrix protein, and the nucleic acid molecule encoding the RNA-dependent RNA polymerase that are carried by the genome of the recombinant oncolytic virus is derived from a Mudd summer subtype virus strain of the vesicular stomatitis virus.

27.-29. (canceled)

30. A pharmaceutical composition, comprising:
the recombinant oncolytic virus according to claim 14.

31. The pharmaceutical composition according to claim 30, wherein the pharmaceutical composition is in a form suitable for inhalation or injection administration.

32.-33. (canceled)

34. A method for preventing or treating a tumor, comprising:
administering, to a subject, the oncolytic virus according to claim 14.

35. (canceled)

36. A method for preventing or treating a tumor, comprising:
administering, to a subject, the pharmaceutical composition according to claim 30.


SEQUENCE LISTING (2 sequences)

SEQ ID NO: 1 - protein, 511 aa, Artificial Sequence
  G protein
MKCFLYLAFLFIGVNCKFTIVFPHNQKGNWKNVPSNYHYCPSSSDLNWHNDLIGTGLQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNDICPTVHNSTTWHSDYKVKGLCDSNLISTDITFFSEDRELSSLGKEGTGFRSNYFAYETGDKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSGLHLSSKAQVFEHPHIQDAASQLPDDEILFFGDTGLSKNPIDFVEGWFSSWKSSIASFFFIIGLIIGLFLVLRVGIYLYIKLKHTKKRQIYTDIEMNRLGR

SEQ ID NO: 2 - protein, 511 aa, Artificial Sequence
  G protein
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK